(19) United States
(12) Patent Application Publication
(10) Pub. No.: US 2024/0069028 A1
(43) Pub. Date: Feb. 29, 2024

(54) MACHINE LEARNING-BASED ASN GROUPING FOR PREDICTING CERVICAL CANCER PROGNOSIS AND CHEMORADIOTHERAPY RESPONSE USING ATP5H, SCP3, AND NANOG

(71) Applicant: INDUSTRY-ACADEMIC COOPERATION FOUNDATION, YONSEI UNIVERSITY, Seoul (KR)

(72) Inventors: Hanbyoul Cho, Seoul (KR); Jae-Hoon Kim, Seoul (KR); Joon-Yong Chung, Rockville, MD (US); Hee Yun, Seoul (KR); Gwan Hee Han, Seoul (KR); Hye Rim Kim, Gyeonggi-do (KR)

(73) Assignee: INDUSTRY-ACADEMIC COOPERATION FOUNDATION, YONSEI UNIVERSITY, Seoul (KR)

(21) Appl. No.: 18/454,527

(22) Filed: Aug. 23, 2023

Related U.S. Application Data
(60) Provisional application No. 63/400,378, filed on Aug. 23, 2022.

(30) Foreign Application Priority Data
Jun. 27, 2023 (KR) 10-2023-0082404

(51) Int. Cl. G01N 33/574

(52) U.S. Cl. CPC G01N 33/57411; G01N 2800/52

(57) ABSTRACT

Provided are biomarkers for predicting the prognosis of cervical cancer. In the case of using the biomarkers of the present disclosure, it is possible to select patients into a high-risk group, an intermediate-risk group, or a low-risk group, and thus, it is possible to provide tailored treatment for each patient according to prognosis prediction.

### CROSS-REFERENCE TO RELATED APPLICATIONS

This application claims the priority of U.S. Provisional Patent Application No. 63/400,378 filed on Aug. 23, 2022, Korean Patent Application No. 10-2023-0082404 filed on Jun. 27, 2023, the disclosure of which is incorporated herein by reference.

### BACKGROUND

### Field

The present disclosure relates to biomarkers for predicting the prognosis of cervical cancer and a method for providing information using the biomarkers.

### Description of the Related Art

Cervical cancer may refer to female genital cancer that occurs in the cervix, which is the entrance to the uterus.

Meanwhile, the diagnosis rate of early cervical cancer has increased due to the development of diagnostic techniques and screening tests, but it is still reported that many patients are diagnosed in the late stage with poor prognosis. Therefore, it may be important to receive an accurate diagnosis and appropriate treatment in time to increase the survival rate of cervical cancer patients.

When cervical cancer is diagnosed, standard treatment guidelines such as surgery, radiation therapy, and chemotherapy have been proposed according to a disease stage based on the National Comprehensive Cancer Network (NCCN) and International Federation of Gynecology and Obstetrics (FIGO) stages. However, even if the patients are treated according to the guidelines, each patient may have a different prognosis due to the heterogeneity of the tumor.

That is, as the accuracy of prognosis prediction has been further required for the improvement of medical services, there is a demand for the development of biomarkers capable of predicting the prognosis of cervical cancer with high accuracy and a method for providing information on prognosis prediction using the biomarkers.

The background art of the invention has been prepared to further facilitate understanding of the present disclosure. It should not be understood that the matters described in the background art of the invention exist as prior arts.

### SUMMARY

Meanwhile, the present inventors paid attention to the fact that several genes closely interact with each other during the development and progression of tumors, and that individual gene expression levels may differ from each other.

Particularly, the present inventors recognized that it is possible to predict the prognosis of cervical cancer for different individuals by using biomarkers capable of predicting the prognosis by evaluating gene expression patterns, thereby enabling tailored personal medicine.

As a result, the present inventors were able to establish genes capable of predicting the prognosis of cervical cancer and determining the treatment direction as biomarkers and confirm clinicopathological relevance of the biomarkers.

Furthermore, the present inventors provided information on prognosis prediction based on the biomarkers, thereby more easily setting the treatment direction for each individual and expecting to contribute to the prognosis improvement.

As a result, the present inventors have developed biomarkers for predicting the prognosis of cervical cancer of the present disclosure.

Accordingly, the present inventors have recognized that the introduction of the biomarkers of the present disclosure can supplement the limitations of conventional diagnosis and information providing systems, and thus, it is possible to present an efficient treatment method.

Accordingly, an object of the present disclosure is to provide biomarkers for predicting the prognosis of cervical cancer through an integrated biomarker of the present disclosure and a method for providing information using the biomarkers.

The objects of the present disclosure are not limited to the aforementioned objects, and other objects, which are not mentioned above, will be apparent to those skilled in the art from the following description.

In order to solve the problems, there is provided biomarkers for predicting the prognosis of cervical cancer according to an exemplary embodiment of the present disclosure.

In an exemplary embodiment of the present disclosure, the biomarkers may include proteins of ATP5H, SCP3, cytoplasmic pERK1/2, NANOG and PTEN or genes encoding the proteins, but are not limited thereto.

In a specific exemplary embodiment of the present disclosure, the biomarkers may include proteins of ATP5H, SCP, and NANOG or genes encoding the proteins, but are not limited thereto.

According to another exemplary embodiment of the present disclosure, there is provided a composition for predicting the prognosis of cervical cancer including a preparation for measuring the expression levels of proteins of ATP5H, SCP, and NANOG or genes encoding the proteins.

In the present disclosure, the preparation for measuring the expression levels of the proteins of ATP5H, SCP, and NANOG may include at least one selected from the group consisting of antibodies, oligopeptides, ligands, peptide nucleic acid (PNA) and aptamers that specifically bind to the proteins of ATP5H, SCP, and NANOG.

In the present disclosure, the preparation for measuring the expression levels of the genes encoding the proteins of ATP5H, SCP, and NANOG may include at least one selected from the group consisting of primers, probes and antisense nucleotides that specifically bind to the genes encoding the proteins of ATP5H, SCP, and NANOG.

According to another exemplary embodiment of the present disclosure, there is provided a kit for predicting the prognosis of cervical cancer including a composition for predicting the prognosis of cervical cancer including a preparation for measuring the expression levels of proteins of ATP5H, SCP, and NANOG or genes encoding the proteins.

In the present disclosure, the kit may be an RT-PCR kit, a DNA chip kit, an ELISA kit, a protein chip kit, a rapid kit, or a multiple reaction monitoring (MRM) kit.

The kit of the present disclosure may further include one or more other component compositions, solutions, or devices suitable for an analysis method. For example, in the present disclosure, the kit may further include essential elements necessary for performing a reverse transcription polymerase reaction. The reverse transcription polymerase reaction kit includes a specific primer pair to a gene encoding the marker protein. The primer is a nucleotide having a specific sequence to the nucleic acid sequence of the gene, and may have a length of about 7 bp to 50 bp, more preferably about 10 bp to 30 bp. In addition, the primer may include a specific primer to a nucleic acid sequence of a control gene. Other reverse transcription polymerase reaction kits may include test tubes or other suitable containers, a reaction buffer (having various pH and magnesium concentrations), deoxynucleotides (dNTPs), enzymes such as Taq-polymerase and reverse transcriptase, DNase, RNase inhibitor DEPC-water, sterilized water, etc.

In addition, the kit for predicting the prognosis of the present disclosure may include essential elements required to perform a DNA chip. The DNA chip kit may include a substrate to which cDNA or oligonucleotides corresponding to genes or fragments thereof are attached, and reagents, agents, enzymes, and the like for preparing fluorescently labeled probes. In addition, the substrate may include cDNA or oligonucleotide corresponding to the control gene or a fragment thereof.

In addition, the kit for predicting the prognosis of the present disclosure may include essential elements required to perform ELISA. The ELISA kit includes a specific antibody to the protein. The antibody is an antibody having high specificity and affinity to the marker protein and little cross-reactivity to other proteins, and includes monoclonal antibodies, polyclonal antibodies, or recombinant antibodies. In addition, the ELISA kit may include a specific antibody to the control protein. Other ELISA kits may include reagents capable of detecting bound antibodies, such as labeled secondary antibodies, chromophores, enzymes (e.g., conjugated with antibodies), and other materials capable of binding the substrates or antibody thereof, and the like.

In the kit for predicting the prognosis of the present disclosure, an immobilizer for an antigen-antibody binding reaction may be used with a nitrocellulose membrane, a PVDF membrane, a well plate synthesized with polyvinyl resin or polystyrene resin, a glass slide made of glass, etc., but is not limited thereto.

In addition, in the kit for predicting the prognosis of the present disclosure, a marker of the secondary antibody is preferably a conventional colorant that undergoes a color reaction, and may be used with markers, such as horseradish peroxidase (HRP), alkaline phosphatase, colloid gold, fluorescein and dyes such as Korean Patent Publication No. 10-2021-0050278-11-poly L-lysine-fluorescein isothiocyanate (FITC) and Rhodamine-B-isothiocyanate (RITC), and the like, but is not limited thereto.

In addition, a chromogenic substrate for inducing color development in the kit for predicting the prognosis of the present disclosure is preferably used according to a marker that undergoes a color reaction, and may be used with 3,3′,5,5′-tetramethylbenzidine (TMB), 2,2′-azino-bis(3-ethylbenzothiazoline-6-sulfonic acid) (ABTS), o-phenylenediamine (OPD), and the like. At this time, it is more preferable that the chromogenic substrate is provided in a state dissolved in a buffer solution (0.1 M NaAc, pH 5.5). A chromogenic substrate such as TMB is decomposed by HRP used as a marker for a secondary antibody conjugate to generate a chromogenic deposit, and the presence or absence of the marker proteins is detected by visually checking the deposition degree of the chromogenic deposit.

In the kit for predicting the prognosis of the present disclosure, a washing solution contains preferably a phosphate buffer solution, NaCl and Tween 20, and more preferably a buffered solution (PBST) consisting of a 0.02 M phosphate buffer solution, 0.13 M NaCl, and 0.05% Tween 20. The washing solution washes the kit 3 to 6 times by reacting the secondary antibody with the antigen-antibody conjugate after the antigen-antibody binding reaction, and then adding an appropriate amount to the immobilizer. As a reaction stop solution, a sulfuric acid solution (H₂SO₄) may be preferably used.

According to another exemplary embodiment of the present disclosure, there is provided a method for providing information for predicting the prognosis of cervical cancer, including measuring the expression levels of at least one protein selected from the group consisting of ATP5H, SCP and NANOG or a gene encoding the protein in a biological sample isolated from a target subject.

At this time, in the present disclosure, the method for providing information for predicting the prognosis of cervical cancer may further include classifying the proteins or the genes into any one of groups 1 to 4 according to the measured expression level, after the measuring of the expression levels of at least one protein selected from the group consisting of ATP5H, SCP and NANOG or the genes encoding the proteins.

According to the feature of the present disclosure, the four groups classified according to the expression level of the biomarker protein are Groups 1 to 4, in which Group 1 may be a group with overexpressed ATP5H (>4), Group 2 may be a group with a low expression level of ATP5H (≤4), Group 3 may be a group with low expression levels of ATP5H (≤4), SCP (≤161) and NANOG (≤185), and Group 4 may be a group with low expression levels of ATP5H (≤4) and SCP (≤161) proteins, and overexpressed NANOG (>185).

In addition, the method may further include providing information on the prognosis prediction by classifying Groups 1 to 4, which are classified according to the expression level of the biomarker, into Group 1 which is a low-risk patient group, Groups 2 and 3 which are intermediate-risk patient groups, and Group 4 which is a high-risk patient group.

In the present disclosure, the preparation for measuring the expression levels of the proteins may include at least one selected from the group consisting of antibodies, oligopeptides, ligands, peptide nucleic acid (PNA) and aptamers that specifically bind to the proteins.

In the present disclosure, the measuring of the expression levels of the proteins may be performed by protein chip analysis, immunoassay, ligand binding assay, matrix assisted laser desorption/ionization time of flight mass spectrometry (MALDI-TOF) analysis, surface enhanced laser desorption/ionization time of flight mass spectrometry (SELDI-TOF) analysis, radioimmunoassay, radioimmunodiffusion method, Ouchterlony immunodiffusion method, rocket immunoelectrophoresis, tissue immunostaining, complement fixation assay, two-dimensional electrophoretic assay, liquid chromatography-mass spectrometry (LC-MS), liquid chromatography-mass spectrometry/mass spectrometry (LC-MS/MS), Western blotting, or enzyme linked immunosorbentassay (ELISA).

In addition, in the present disclosure, the expression level of the protein may be measured by a multiple reaction monitoring (MRM) method.

In the MRM method of the present disclosure, an internal standard material may be used with a synthetic peptide or E. coli beta-galactosidase in which a specific amino acid constituting a target peptide is substituted with an isotope.

In the present disclosure, the preparation for measuring the expression levels of the genes encoding the proteins may include at least one selected from the group consisting of primers, probes and antisense nucleotides that specifically bind to the genes encoding the proteins.

In the present disclosure, the measuring of the expression levels of the genes encoding the proteins may be performed by reverse transcription polymerase reaction (RT-PCR), competitive reverse transcription polymerase reaction (Competitive RT-PCR), real-time reverse transcription polymerase reaction (Real-time RTPCR), RNase protection assay (RPA), Northern blotting, or DNA chips.

In the present disclosure, when the expression levels of the proteins or the genes encoding the proteins measured for the biological sample of the target subject increase or decrease compared to those of a control group, it may be predicted that the prognosis after treatment for the cervical cancer is poor.

At this time, in the present disclosure, the method for providing information for predicting the prognosis of cervical cancer may further include providing results for predicting the prognosis of cervical cancer for the subject according to the classified groups, after measuring the expression levels of at least one protein selected from the group consisting of ATP5H, SCP and NANOG or genes encoding the proteins, and classifying the proteins or the genes into any one of Groups 1 to 4 according to the measured expression levels.

At this time, the providing of the results for predicting the prognosis of cervical cancer for the subject according to the classified groups may further include providing results for predicting the prognosis of cervical cancer for the subject by including the expression levels of at least one protein selected from the group consisting of ATP5H, SCP and NANOG or genes encoding the proteins in the biological sample isolated from the subject and clinical data information on the subject. In one exemplary embodiment, the clinical data may be data on FIGO stage, tumor size, lymph node metastasis, oncological grade, and age, but are not limited thereto.

Details of other exemplary embodiments will be included in the detailed description of the disclosure and the accompanying drawings.

According to the present disclosure, it is possible to provide biomarkers for predicting the prognosis of cervical cancer and a method for providing information using the biomarkers.

Particularly, according to the present disclosure, it is possible to enable to treat with a tailored personal medicine for different individuals by providing the biomarkers capable of predicting the prognosis of cervical cancer to predict the prognosis after treatment of cervical cancer.

That is, according to the present disclosure, it is possible to perform more easily setting of the treatment direction for each individual and to contribute to the selection of a tailored treatment method by using the biomarkers capable of predicting the prognosis of cervical cancer.

The effects of the present disclosure are not limited by the foregoing, and other various effects are anticipated herein.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 exemplarily illustrates a procedure of a method for providing information on the prognosis of cervical cancer according to an exemplary embodiment of the present disclosure.

FIG. 2 illustrates results of ranking the top 11 biomarkers associated with recurrence of cervical cancer using a random forest algorithm.

FIGS. 3 and 4 illustrate expression patterns for each group classified according to expression levels of biomarkers used in various Examples of the present disclosure.

FIG. 5 is a diagram illustrating progression free survival (PFS) using Kaplan-Meier survival analysis.

FIG. 6 illustrates evaluation results for prognosis prediction of biomarkers used in various Examples of the present disclosure.

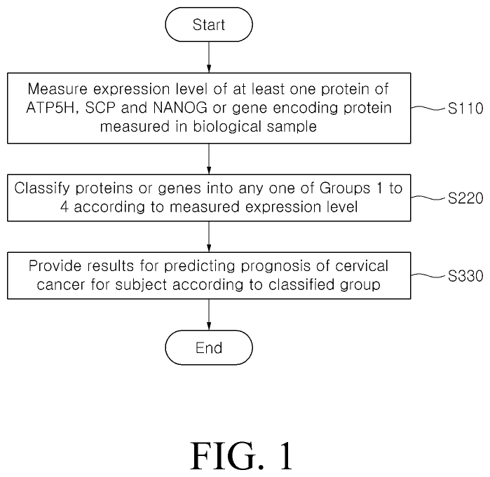

### DETAILED DESCRIPTION OF THE EMBODIMENT

Advantages and features of the present disclosure, and methods for accomplishing the same will be more clearly understood from exemplary embodiments to be described below in detail with reference to the accompanying drawings. However, the present disclosure is not limited to the exemplary embodiments set forth below, and will be embodied in various different forms. The exemplary embodiments are just for rendering the disclosure of the present disclosure complete and are set forth to provide a complete understanding of the scope of the invention to a person with ordinary skill in the art to which the present disclosure pertains, and the present disclosure will only be defined by the scope of the claims. In connection with the description of the drawings, like reference numerals may be used for like components.

In this document, expressions such as “have,” “may have,” “include,” or “may include” indicate the existence of a corresponding feature (e.g., numerical value, function, operation, or component such as a part), and do not exclude the existence of additional features.

In the present document, the expression such as “A or B”, “at least one of A and/or B”, or “one or more of A and/or B” may include all possible combinations of items listed together. For example, “A or B”, “at least one of A and B”, or “at least one of A or B” may refer to all cases of (1) including at least one A, (2) including at least one B, or (3) including both at least one A and at least one B.

Expressions such as “first,” and “second,” used herein may modify various components regardless of the order and/or importance, and will be used only to distinguish one component from the other component, but are not limit the corresponding components. For example, a first user device and a second user device may represent different user devices, regardless of the order or importance. For example, a first component may be referred to as a second component, and similarly, the second component may also be referred to as the first component without departing from the scope of the present document.

When a certain component (e.g., a first component) is referred to as being “(operatively or communicatively) coupled with/to” or “connected to” the other component (e.g., a second component), it will be understood that the component may be directly connected to the other component, or may be connected to the other component through another component (e.g., a third component). On the contrary, when a certain component (e.g., a first component) is referred to as being “directly coupled with/to” or “directly connected to” the other component (e.g., a second component), it will be understood that another component (e.g., a third component) is not present between the certain component and the other component.

The expression of “configured to (or set to)” used herein may be changed and used to, for example, “suitable for”, “having the capacity to”, “designed to”, “adapted to”, “made to” or “capable of”, depending on the situation. The term of “configured to (or set to)” may not necessarily mean only “specially designed to” in hardware. In some situations, the expression “a device configured to” may mean that the device is “capable of” together with other devices or parts. For example, the phrase “a processor configured (or set) to perform A, B, and C” may mean a dedicated processor (e.g., an embedded processor) for performing the corresponding operation, or a generic-purpose processor (e.g., a CPU or application processor) capable of performing the corresponding operations by executing one or more software programs stored in a memory device.

The terms used herein are used to illustrate only specific exemplary embodiments, and may not be intended to limit the scope of other exemplary embodiments. A singular form may include a plural form unless otherwise clearly meant in the contexts. The terms used herein, including technical or scientific terms, may have the same meaning as generally understood by those of ordinary skill in the art described in the present document. The terms defined in a general dictionary among the terms used herein may be interpreted in the same or similar meaning as or to the meaning on the context of the related art, and will not be interpreted as an ideal or excessively formal meaning unless otherwise defined in the present document. In some cases, even the terms defined in the present document cannot be interpreted to exclude the exemplary embodiments of the present disclosure.

The features of various exemplary embodiments of the present disclosure can be partially or entirely coupled or combined with each other and can be interlocked and operated in technically various ways to be sufficiently appreciated by those skilled in the art, and the exemplary embodiments can be implemented independently of or in association with each other.

For the clarity of the interpretation of the present specification, hereinafter, the terms used in the present specification will be defined.

As used herein, the term “subject” may mean any subject for which the prognosis of cervical cancer is to be predicted. For example, the subject may be a subject with cervical cancer. At this time, the subject disclosed in the present specification may be any mammal except for humans, but is not limited thereto.

As used herein, the term “biological sample” refers to any material, biological fluid, tissue, or cell obtained from the subject or derived from the subject. The biological sample may be at least one selected from the group consisting of whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract and cerebrospinal fluid. Preferably, the biological sample may be cervical cancer tissue (cells) isolated from a subject.

As used herein, the term “biomarker” is a biological indicator that may determine internal changes using cells, blood vessels, proteins, DNA, RNA, metabolites, etc. in the body, and the National Institutes of Health (NIH) has defined the biomarker as an index capable of objectively measuring and evaluating normal biological processes, disease progression conditions, and drug responsiveness to treatment methods. That is, in the case of a specific disease or cancer, the biomarker refers to a marker capable of distinguishing between normal and pathological conditions, or predicting a treatment response and objectively measuring the treatment response. Accordingly, the biomarkers should serve to objectively measure and evaluate the normal biological processes, disease progression situations, and drug responsiveness to treatment methods. Depending on applications, the biomarkers include target markers to confirm the existence of drug targets, diagnostic markers to diagnose the presence or absence of diseases, predictive markers to distinguish between responders and non-responders to a specific drug, and surrogate markers to monitor drug treatment effects, prognosis biomarkers to inform the prognosis of diseases, and the like.

As used herein, the term “prognosis” refers to the act of predicting the course of a disease and the outcome of death or survival in advance. The prognosis or prognosis prediction may be interpreted to mean all actions that the course of the disease may vary depending on physiological or environmental conditions of the patient, and the course of the disease before/after treatment is predicted by comprehensively considering the patient's condition.

As used herein, the term “antibody” refers to a substance that specifically binds to an antigen and causes an antigen-antibody reaction. For the purpose of the present disclosure, the antibody refers to an antibody that specifically binds to each protein of ATP5H, SCP and NANOG. The antibody of the present disclosure includes all polyclonal antibodies, monoclonal antibodies and recombinant antibodies. The antibodies may be easily prepared using techniques well-known in the art. For example, the polyclonal antibodies may be produced by a method well known in the art, including a process of injecting an antigen of the protein into an animal and collecting blood from the animal to obtain serum containing the antibody. These polyclonal antibodies may be prepared from any animal such as goat, rabbit, sheep, monkey, horse, pig, cow, dog, and the like. In addition, the monoclonal antibodies may be prepared using a hybridoma method well-known in the art (see Kohler and Milstein (1976) European Journal of Immunology 6:511-519), or a phage antibody library technique (see Clackson et al, Nature, 352:624-628, 1991; Marks et al, J. Mol. Biol., 222:58, 1-597, 1991). The antibodies prepared by the method may be separated and purified using methods such as gel electrophoresis, dialysis, salt precipitation, ion exchange chromatography, and affinity chromatography. In addition, the antibodies of the present disclosure include functional fragments of antibody molecules as well as complete forms having two full-length light chains and two full-length heavy chains. The functional fragments of the antibody molecules refer to fragments having at least an antigen binding function, and may be Fab, F(ab′), F(ab′)2, Fv and the like.

As used herein, the term “oligopeptide” consists of 2 to amino acids as a peptide and may include dipeptide, tripeptide, tetrapeptide and pentapeptides, but is not limited thereto.

As used herein, the term “peptide nucleic acid (PNA)” refers to an artificially synthesized polymer similar to DNA or RNA, and was first introduced in 1991 by Professors Nielsen, Egholm, Berg and Buchardt of the University of Copenhagen, Denmark. While DNA has a phosphate-ribose backbone, PNA has a repeated N-(2-aminoethyl)-glycine backbone linked by peptide bonds to greatly increase the binding force and stability to DNA or RNA, and accordingly, has been used in molecular biology, diagnostic assay and antisense therapies.

As used herein, the term “aptamer” means an oligonucleic acid or peptide molecule.

In the present disclosure, the preparation for measuring the expression levels of the genes encoding the proteins of ATP5H, SCP, and NANOG may include at least one selected from the group consisting of primers, probes and antisense nucleotides that specifically bind to the genes encoding the proteins of ATP5H, SCP, and NANOG.

As used herein, the term “primer” is a fragment that recognizes a target gene sequence, and includes a pair of forward and reverse primers, preferably a primer pair that provides an analysis result having specificity and sensitivity. Since the nucleic acid sequence of the primer is a sequence that is inconsistent with a non-target sequence present in a sample, high specificity may be imparted in the case of a primer in which only a target gene sequence containing a complementary primer binding site is amplified and non-specific amplification is not caused.

As used herein, the term “probe” means a substance capable of specifically binding to a target substance to be detected in a sample, and a substance capable of specifically confirming the presence of the target substance in the sample through the binding. The type of probe is not limited as any material commonly used in the art, but may be preferably peptide nucleic acid (PNA), locked nucleic acid (LNA), peptide, polypeptide, protein, RNA or DNA, and most preferably PNA. More specifically, the probe is a biomaterial, which is derived from or similar to a living organism or produced in vitro, and may be, for example, enzymes, proteins, antibodies, microorganisms, animal and plant cells and organs, neurons, DNA, and RNA. DNA includes cDNA, genomic DNA, and oligonucleotides, RNA includes genomic RNA, mRNA, and oligonucleotides, and examples of proteins may include antibodies, antigens, enzymes, peptides, and the like.

As used herein, the term “locked nucleic acids (LNA)” means a nucleic acid analog containing a 2′-O, 4′-C methylene bridge. The LNA nucleoside includes common nucleic acid bases of DNA and RNA, and may form base pairs according to the Watson-Crick base pairing rules. However, due to the ‘locking’ of the molecule by the methylene bridge, the LNA does not form an ideal shape in the Watson-Crick bond. When the LNA is included in DNA or RNA oligonucleotide, the LNA may more rapidly pair with a complementary nucleotide chain to increase the stability of a double helix.

As used herein, the term “antisense” refers to an oligomer having a sequence of nucleotide bases and a backbone between subunits in which an antisense oligomer hybridizes with a target sequence in RNA by Watson-Crick base pairing to typically allow the formation of heteroduplex of mRNA and RNA:oligomer in the target sequence. The oligomer may have exact sequence complementarity or approximate complementarity to the target sequence.

Since the information of the proteins of ATP5H, SCP and NANOG or the genes encoding the proteins according to the present disclosure is known, based on this, those skilled in the art may easily design primers, probes, or antisense nucleotides that specifically bind to the genes encoding the proteins.

As used herein, the term “kit” refers to a tool capable of evaluating the expression level of a biomarker by labeling a probe or antibody that specifically binds to a biomarker component with a detectable label. The kit includes not only direct labeling of a detectable substance in relation to the probe or antibody by reaction with a substrate, but also indirect labeling in which a color-developing marker is conjugated by reactivity with another directly labeled reagent. The kit may include a color-developing substrate solution to color-react with the marker, a washing solution, and other solutions, and may be prepared by including reagent components to be used. In the present disclosure, the kit may be a kit including essential elements necessary for performing RT-PCR, and may include a test tube, a reaction buffer, deoxynucleotides (dNTPs), Taq-polymerase, reverse transcriptase, DNase, RNase inhibitors, sterile water, and the like in addition to each pair of primers specific to the marker gene. In addition, the kit may be a kit for detecting a gene for predicting HPD prognosis including essential elements necessary for performing DNA chip. The DNA chip kit may include a substrate to which cDNA corresponding to a gene or a fragment thereof is attached as a probe, and the substrate may include cDNA corresponding to a quantitative control gene or a fragment thereof. The kit of the present disclosure is not limited thereto, as long as the kit is known in the art.

As used herein, the term “subject data” is data obtainable from a subject, and may include at least one of cancer stage, lymph node metastasis, and age.

However, the subject data is not limited thereto, and may include more various data obtainable from electronic medical record data.

Hereinafter, the present disclosure will be described in more detail through Examples. These Examples are to explain the present disclosure in more detail, and it will be apparent to those skilled in the art that the scope of the present disclosure is not limited by these Examples in accordance with the gist of the present disclosure.

Evaluation 1: Derivation of Biomarkers and Performance Evaluation

First, referring to Table 1, Table 1 shows a classification result of patient groups for deriving biomarkers of the present disclosure.

TABLE 1

| | All | | Test cohort | | Training cohort | | |
|---|---|---|---|---|---|---|---|
| | (N = 281) | | (N = 89) | | (N = 192) | | P value |
| Age, years old | 49.37 ± 11.40 | | 50.55 ± 10.78 | | 48.82 ± 11.66 | | 0.236 |
| FIGO stage | | | | | | | 0.363 |
| I-IIA | 190 | (67.62%) | 64 | (71.91%) | 126 | (65.62%) | |
| IIB-IV | 91 | (32.38%) | 25 | (28.09%) | 66 | (34.38%) | |
| Cell Type | | | | | | | 0.097 |
| Squamous | 229 | (81.49%) | 67 | (75.28%) | 162 | (84.38%) | |
| Others | 52 | (18.51%) | 22 | (24.72%) | 30 | (15.62%) | |
| Grade | | | | | | | 0.188 |
| Low/Moderate | 158 | (60.77%) | 57 | (67.06%) | 101 | (57.71%) | |
| Poor | 102 | (39.23%) | 28 | (32.4%) | 74 | (42.29%) | |
| Tumor size (mm) | 30.54 ± 19.40 | | 28.54 ± 17.04 | | 31.47 ± 20.38 | | 0.24 |
| LVI | | | | | | | 0.934 |
| Negative | 95 | (51.35%) | 28 | (50.00%) | 67 | (51.94%) | |
| Positive | 90 | (48.65%) | 28 | (50.00%) | 62 | (46.06%) | |
| Lymph node metastasis | | | | | | | 0.911 |
| Negative | 188 | (73.68%) | 50 | (72.46%) | 118 | (74.21%) | |
| Positive | 60 | (26.32%) | 19 | (27.54%) | 41 | (25.79%) | |

SCC antigen

| Response to Chemoradiation therapy | | | | | | | 0.283 |
|---|---|---|---|---|---|---|---|
| Good | 81 | (57.45%) | 31 | (64.58%) | 50 | (53.76%) | |
| Poor | 60 | (42.55%) | 17 | (35.42%) | 43 | (46.24%) | |

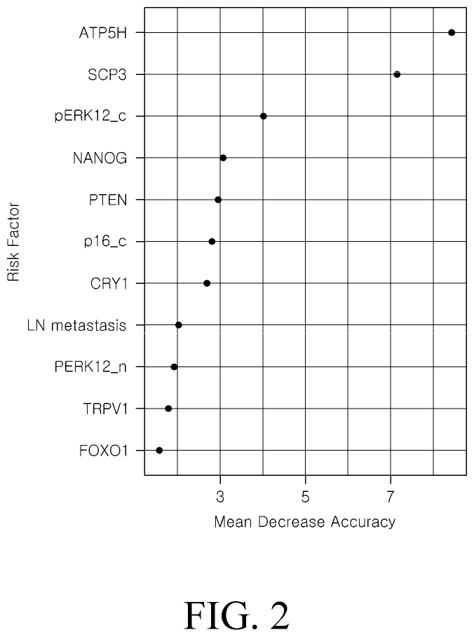

For the derivation of biomarkers for predicting the prognosis of cervical cancer, as shown in Table 1, patient groups were divided into a learning group (70%) and a verification group (30%), and then as illustrated in FIG. 2, the top 11 biomarkers associated with recurrence of cervical cancer were ranked using a random forest algorithm. Thereafter, considering the significance of the biomarkers, 5 major biomarkers consisting of ATP5H, SCP3, cytoplasmic pERK1/2, NANOG and PTEN were selected.

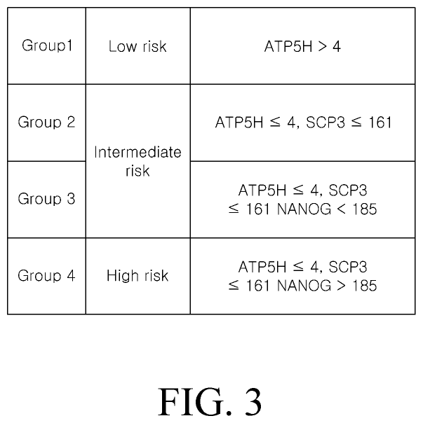

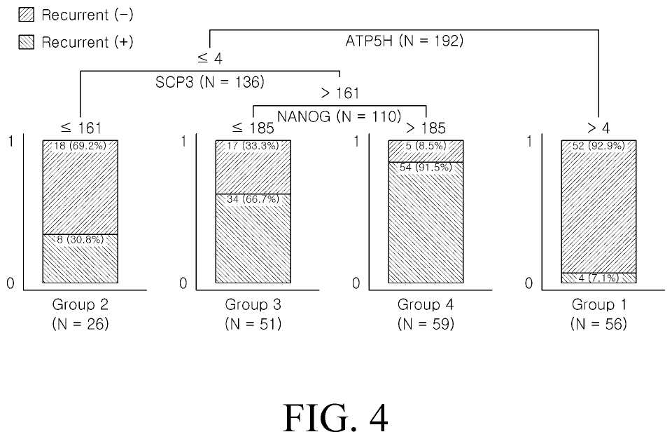

At this time, referring to FIGS. 3 and 4 together, the results of dividing the risk of recurrence of cervical cancer into 4 groups based on the expression levels of biomarkers ATP5H, SCP3, cytoplasmic pERK1/2, NANOG and PTEN were illustrated.

Group 1 was a low-risk group, in which ATP5H was expressed higher than a predetermined cutoff value (ATP5H>4). Groups 2 and 3 were intermediate-risk groups. First, in Group 2, it was confirmed that both ATP5H and SCP3 were expressed lower than the cutoff value (ATP5H≤4, SCP≤161), and in Group 3, it was confirmed that ATP5H, SCP3, and NANOG all were expressed lower than the cutoff value (ATP5H≤4, SCP≤161, and NANOG≤185). Group 4 was a high-risk group, and it was confirmed that ATP5H and SCP3 were expressed lower than the cutoff value, but the expression of NANOG was higher than the cutoff value (ATP5H≤4, SCP≤161, NANOG>185).

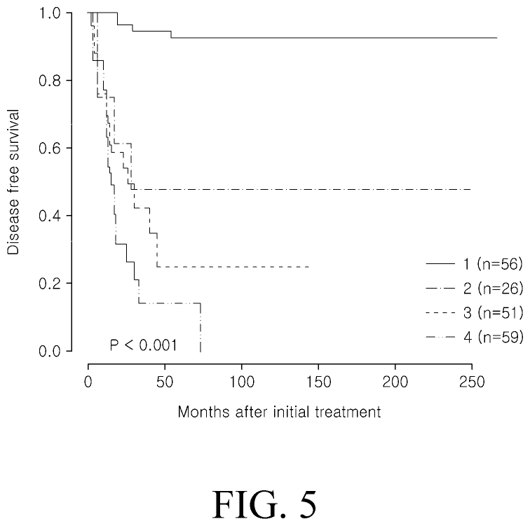

Referring to FIG. 5 together, FIG. 5 is a diagram illustrating progression free survival (PFS) using Kaplan-Meier survival analysis, and it was confirmed that in both the learning and verification groups in Group 1 as the low-risk group, Groups 2 and 3 as the intermediate-risk groups, and Group 4 as the high-risk group, there were clear differences among the low, intermediate, and high-risk groups. This indicated that the performance of the biomarkers for predicting recurrence of cervical cancer of the present disclosure was excellent.

Based on the results, when using the biomarkers of the present disclosure, it is possible to predict the prognosis of cervical cancer with high accuracy, so that it is expected to contribute to increasing the life expectancy of patients by providing customized treatment tailored to each patient.

Evaluation 2: Evaluation of Biomarkers

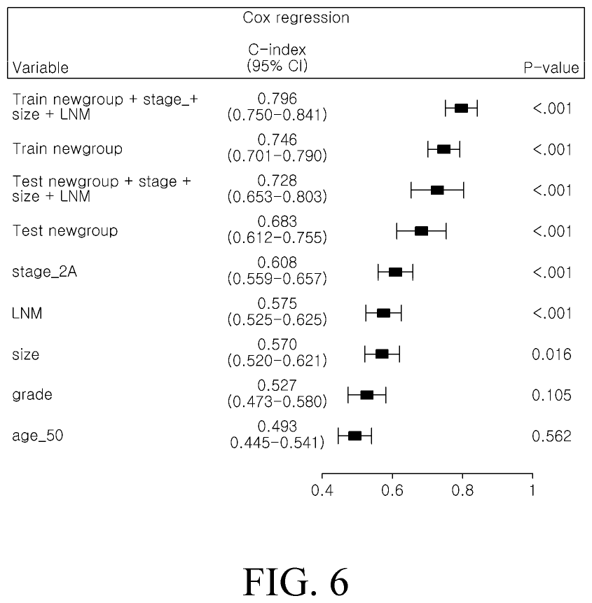

Hereinafter, evaluation results of biomarkers for predicting the prognosis of cervical cancer according to various Examples of the present disclosure will be described with reference to FIG. 6 and Table 2. Table 3 shows results of analyzing clinicopathological characteristics by group. FIG. 6 illustrates evaluation results for prognosis prediction of biomarkers used in various Examples of the present disclosure.

TABLE 2

| | Group1 | Group2 | Group3 | Group4 | |
|---|---|---|---|---|---|
| | [symbol] | [symbol] | [symbol] | [symbol] | P-value |
| Age | [symbol] | [symbol] | [symbol] | [symbol] | [symbol] |
| FIGO stage | | | | | [symbol] |
| I-IIA | 52 [symbol] | 22 [symbol] | [symbol] | 27 [symbol] | |
| IIB-IV | 4 [symbol] | 4 [symbol] | [symbol] | [symbol] | |
| Cell Type | | | | | [symbol] |
| Squamous | [symbol] | 19 [symbol] | 44 [symbol] | [symbol] | |
| Others | 8 [symbol] | 7 [symbol] | 7 [symbol] | 7 [symbol] | |
| Grade | | | | | [symbol] |
| Low/Moderate | 31 [symbol] | [symbol] | [symbol] | 37 [symbol] | |
| Poor | 17 [symbol] | [symbol] | 22 [symbol] | 27 [symbol] | |
| Tumor size (mm) | [symbol] | 22.42 | [symbol] | [symbol] | [symbol] |
| LVI | | | | | [symbol] |
| Negative | [symbol] | [symbol] | [symbol] | [symbol] | |
| Positive | 15 [symbol] | [symbol] | [symbol] | 24 [symbol] | |

Lymph node metastasis

| | | | | | |
|---|---|---|---|---|---|
| Negative | 45 [symbol] | [symbol] | 18 [symbol] | [symbol] | [symbol] |
| Positive | 7 [symbol] | [symbol] | [symbol] | [symbol] | |

Response to Chemoradiation therapy

| | | | | | |
|---|---|---|---|---|---|
| Good | 13 [symbol] | [symbol] | [symbol] | [symbol] | [symbol] |
| Poor | 1 [symbol] | [symbol] | [symbol] | [symbol] | |

[symbol]

indicates data missing or illegible when filed

Referring to Table 2, it was confirmed that the FIGO stage increased significantly from Group 1 to Group 4.

In Group 1, the low-risk group, a ratio of subjects classified as FIGO stage IIB-IV was only 7.14%, but in Group 4, the high-risk group, it was confirmed that the ratio of subjects increased to 52.24%. It was confirmed that the tumor size also increased significantly from Group 1, the low-risk group to Group 4, the high-risk group, and the size increased from 21.93±20.31 mm to 40.25±17.21 mm.

In addition, in Group 4, lymph node metastasis was confirmed as 61.54%, and the ratio of subjects with poor treatment response was 59.62%, which was higher than those of Groups 1 to 3.

In the case of LVSI, it was confirmed that the expression rate was high in Groups 3 and 4 compared to other groups, and specifically, 80% in Group 3 and 60% in Group 4.

Referring to Table 3, Table 3 was a table comparing Groups 3 and 4.

TABLE 3

| | Group2 | Group3 | |
|---|---|---|---|
| | (N = 26) | (N = 51) | P value |
| Age | [symbol] 3.92 ± 11.29 | 47.38 ± 30.15 | 0.027 |
| FIGO stage | | | <0.001 |
| I-IIA | 22 (84.62%) | 25 (49.51%) | |
| IIB-IV | 4 (15.38%) | 26 (50.98%) | |
| Cell Type | | | 0.099 |
| Squamous | 19 (75.1%) | 44 (36.3%) | |
| Others | 7 (26.9%) | 7 (17.7%) | |
| Grade | | | 0.729 |
| Low/Moderate | 14 (63.64%) | 29 (56.36%) | |
| Poor | [symbol] (36.36%) | 22 (43.54%) | |
| Tumor size (mm) | 22.42 ± 16.62 | 26.92 ± 19.48 | <0.001 |
| LVI | | | 0.044 |
| Negative | 15 (57.7%) | 3 (20.0%) | |
| Positive | 11 (42.3%) | 12 (80.0%) | |
| LN metastasis | | | 0.050 |
| Negative | 23 (81.46%) | 18 (62.07%) | |
| Positive | 3 ( [symbol] ) | 11 (37.93%) | |
| Response to chemoradiation therapy | | | 0.018 |
| Good | 8 (100.00%) | 11 (57.89%) | |
| Poor | 0 (0.0%) | [symbol] (42.11%) | |

[symbol]

indicates data missing or illegible when filed

As shown in Table 3, subjects in Group 3 were younger at the time of diagnosis, diagnosed as more advanced in the FIGO stage, and had larger tumor sizes and higher LVSI expression rate than those of Group 2.

As described above, according to the classification model of the present disclosure, it is possible to classify subjects according to the risk of cervical cancer, and according to the tumor size, lymph node metastasis, FIGO stage and LVSI for each group, it is possible to develop a tailored treatment plan appropriate for the patient and to more accurately predict the prognosis of patients, and thus it was confirmed the present disclosure can be usefully used to improve the prognosis of cervical cancer patients.

Hereinafter, evaluation results of a classification model according to various Examples of the present disclosure will be described with reference to FIG. 6.

FIG. 6 illustrates evaluation results of a classification model used for various Examples of the present disclosure. Specifically, clinical information, such as FIGO stage, tumor size, lymph node metastasis, oncological grade, and age, was integrated into the classification model of the present disclosure to be classified by combining molecular information such as expression levels of biomarkers, and C-indexes were compared by comparing a case of considering only clinical information and a clinical-molecular classification using the classification model of the present disclosure.

As a result, as illustrated in FIG. 6, it was confirmed that it is possible to predict the prognosis of cervical cancer with higher accuracy when using a model considering both molecular classification and clinical information than when the molecular classification and the clinical information were considered separately.

Although the exemplary embodiments of the present disclosure have been described in detail with reference to the accompanying drawings, the present disclosure is not limited thereto and may be embodied in many different forms without departing from the technical concept of the present disclosure. Therefore, the exemplary embodiments of the present disclosure are provided for illustrative purposes only but not intended to limit the technical concept of the present disclosure. The scope of the technical concept of the present disclosure is not limited thereto. Therefore, it should be appreciated that the aforementioned exemplary embodiments are illustrative in all aspects and are not restricted. The protective scope of the present disclosure should be construed on the basis of the appended claims, and all the technical spirits in the equivalent scope thereof should be construed as falling within the scope of the present disclosure.

What is claimed is:

1. Biomarkers for predicting prognosis of cervical cancer comprising at least one protein of ATP5H, NANOG, PTEN, SCP and pERK or genes encoding the proteins.

2. The biomarkers for predicting the prognosis of cervical cancer of claim 1, wherein the biomarkers are ATP5H, SCP and NANOG.

3. A composition for predicting prognosis of cervical cancer comprising a preparation for measuring expression levels of proteins of ATP5H, SCP, and NANOG or genes encoding the proteins.

4. The composition for predicting the prognosis of cervical cancer of claim 3, wherein the preparation for measuring the expression levels of the proteins of ATP5H, SCP, and NANOG includes at least one selected from the group consisting of antibodies, oligopeptides, ligands, peptide nucleic acid (PNA) and aptamers that specifically bind to the proteins of ATP5H, SCP, and NANOG.

5. The composition for predicting the prognosis of cervical cancer of claim 3, wherein the preparation for measuring the expression levels of the genes encoding the proteins of ATP5H, SCP, and NANOG includes at least one selected from the group consisting of primers, probes and antisense nucleotides that specifically bind to the genes encoding the proteins of ATP5H, SCP3, and NANOG.

6. A method for providing information for predicting prognosis of cervical cancer, comprising measuring expression levels of at least one protein selected from the group consisting of ATP5H, SCP and NANOG or genes encoding the proteins in a biological sample isolated from a subject.

7. The method for providing information for predicting the prognosis of cervical cancer of claim 6, further comprising:
classifying the proteins or the genes into any one of groups 1 to 4 according to the measured expression level.

8. The method for providing information for predicting the prognosis of cervical cancer of claim 7, further comprising:
providing results for predicting the prognosis of cervical cancer for the subject according to the classified groups.

9. The method for providing information for predicting the prognosis of cervical cancer of claim 8, wherein in the providing of the results for predicting the prognosis of cervical cancer for the subject according to the classified groups,
the group 1 is a group with overexpressed ATP5H as compared with a normal control group.

10. The method for providing information for predicting the prognosis of cervical cancer of claim 8, wherein in the providing of the results for predicting the prognosis of cervical cancer for the subject according to the classified groups,
the group 2 is a group with low-expressed ATP5H as compared with a normal control group.

11. The method for providing information for predicting the prognosis of cervical cancer of claim 8, wherein in the providing of the results for predicting the prognosis of cervical cancer for the subject according to the classified groups,
the group 3 is a group with low-expressed ATP5H, SCP, and NANOG.

12. The method for providing information for predicting the prognosis of cervical cancer of claim 8, wherein in the providing of the results for predicting the prognosis of cervical cancer for the subject according to the classified groups,
the group 4 is a group with low-expressed ATP5H and SCP, and overexpressed NANOG.

13. The method for providing information for predicting the prognosis of cervical cancer of claim 8, further comprising:
classifying the group 1 as a low-risk group.

14. The method for providing information for predicting the prognosis of cervical cancer of claim 8, further comprising:
classifying the groups 2 and 3 as intermediate-risk groups.

15. The method for providing information for predicting the prognosis of cervical cancer of claim 8, further comprising:
classifying the group 4 as a high-risk group.

16. The method for providing information for predicting the prognosis of cervical cancer of claim 6, wherein the providing of the results for predicting the prognosis of cervical cancer for the subject according to the classified groups further includes
providing results for predicting the prognosis of cervical cancer for the subject by including the expression levels of at least one protein selected from the group consisting of ATP5H, SCP and NANOG or genes encoding the proteins in the biological sample isolated from the subject and clinical data information on the subject.

17. The method for providing information for predicting the prognosis of cervical cancer of claim 16, wherein the clinical data is data on FIGO stage, tumor size, lymph node metastasis, oncological grade, and age.

18. A kit for predicting prognosis of cervical cancer comprising the biomarkers of claim 1.
